(19) United States
(12) Patent Application Publication
(10) Pub. No.: US 2007/0233145 A1
(43) Pub. Date: Oct. 4, 2007

(54) TENSEGRITY OSTEOTOMY SYSTEM

(75) Inventors: James Bruce Richardson, Oswestry (GB); John Joseph Cooper, Crewe (GB); Russell David Waters, Knutsford (GB)

Correspondence Address: SMITH-HILL AND BEDELL, P.C., 16100 NW CORNELL ROAD, SUITE 220, BEAVERTON, OR, 97006, US

(73) Assignee: BIOCOMPOSITES LTD., Keele (GB)

(21) Appl. No.: 11/675,205

(22) Filed: Feb. 15, 2007

(30) Foreign Application Priority Data
Aug. 18, 2005 (GB) 0516911.5
Aug. 16, 2006 (GB) 0616271.3

(51) Int. Cl. A61B 17/58

(52) U.S. Cl. 606/90

(57) ABSTRACT

A method of carrying out an opening wedge osteotomy, the method including forming a cut in the bone, locating inserts in guide holes formed in the cut, and applying a closing force to either side of the cut using a screw, plate or tension bands extending across the opening of the cut, to ensure compressive and tensile forces are developed across the cut.

### BACKGROUND OF THE INVENTION

This invention concerns apparatus for use in opening wedge osteotomy, and also a method of carrying out an opening wedge osteotomy.

An osteotomy is a surgical procedure whereby a bone is partially or completely cut through, then realigned or repositioned and held in its new position where it is allowed to fuse together and unite. It is a technique that is used to correct skeletal deformity arising either through trauma, disease or congenital defects. The type of osteotomy performed depends upon the type of skeletal correction required.

For realignment of the angle of a bone, as opposed to a translation or axial or rotational realignment, the type of osteotomy can be either an opening or closing wedge. In a closing wedge procedure, two convergent cuts are made to form a bone wedge that is then removed. Bending one part of the bone relative to the other then closes the resultant gap and the angle of correction is equal to the taper angle of the bone wedge removed. In an opening wedge procedure, a single cut is made through the bone. Bending one part of the bone relative to the other opens up a gap that is maintained by some means while healing occurs. The bone is not cut completely through so that the far cortex can act as a hinge to the bending bone.

Unicompartmental arthritis of the knee with associated pain and instability is traditionally treated by total knee arthroplasty (TKA). This is a successful procedure with a survivorship of typically over 90% at 10 years. Revision procedures are, however, becoming increasingly common, and for the younger and more active patient undergoing TKA, further revisions may be required later in life.

High tibial or distal femoral opening wedge osteotomy procedures can be performed in cases of unicompartmental arthritis of the knee. They are far less invasive procedures than TKA and can be revised to a TKA later in life when the patient is older and generally less active. These procedures work by shifting the load axis from the damaged compartment to the healthy or less damaged side of the knee. To enable early weight bearing and to provide an environment conducive to healing, adequate post-operative stability is essential.

In an opening wedge osteotomy procedure, maintenance of the gap between the cut ends of bone is generally undertaken by the use of a metallic plate that bridges the gap and is attached to the bones with surgical screws. A load-bearing device, such as a metallic block, is often used within the cortical gap to prevent gap closure and help support the compressive forces involved in weight bearing. The use of a single block, however, to support compression is inherently unstable since the loads involved in weight bearing are then only supported at two points (the block and the apex of the cut i.e. the cortical hinge). This will allow cyclic micromovements to occur which are not the favored biomechanical environment for the maturation of bone or cancellous bone healing.

Alternatively external fixators may be used. These have the advantage of allowing slow opening of the gap and this encourages new bone formation. Unfortunately these external fixators are not well tolerated by the patient and pin-tract infection is often a complication which occasionally becomes serious.

Another prior system uses an absorbable wedge to fill the osteotomy. This wedge provides only limited stability and does not permit controlled introduction into the space. By occupying the space in the osteotomy it is not possible to introduce other components, such as bone graft materials, into this space.

Tensegrity is a system that confers mechanical stability to a structure by the combined use of the counteracting forces of compression and tension in different elements of the structure.

### SUMMARY OF THE INVENTION

According to the present invention there is provided apparatus for use in opening wedge osteotomy, the apparatus including an insert locatable in an opening in bone, the insert being elongate and converging to its distal end, the insert having a helical external rib, and a guide formation at its proximal end to facilitate rotation thereof.

The insert may be substantially conical or frusto-conical.

The insert may be conical and may taper from its proximal end to its distal end, and the degree of tapering may increase towards the distal end. The distal end of the insert may be substantially bell shaped.

The insert may be made of an osteoconductive and/or bioabsorbable material.

The guide formation may comprise a profiled opening in the proximal end of the insert, and the opening may be hexagonal or cruciform

The external rib may finish spaced from the proximal end of the insert.

The apparatus preferably includes a pair of inserts.

The apparatus preferably also includes gap closing means for applying a compressive force across the wedge gap.

The gap closing means may include a plate or plates locatable across the opening of the wedge gap.

The gap closing means may include a screw or screws extendable across the wedge gap.

The gap closing means may include tension bands extendable across the opening of the wedge gap. Pins and/or screws may be provided mountable on either side of the wedge gap, between which the tension bands may extend.

The invention also provides a method of carrying out an opening wedge osteotomy, the method including forming a cut in the bone, forming two guide holes in the cut plane, locating in each guide hole an insert as defined above, moving the inserts into the cut plane to open same, and applying a closing force to either side of the cut.

The two guide holes are preferably convergent.

The closing force may be provided by locating a plate or plates across the opening of the cut.

The closing force may be provided by locating a screw or screws extendible across the cut, which screw or screws is preferably inclined relative to the cut.

The closing force may be provided by extending tension bands across the opening. Pins and/or screws may be mounted in the bone on either side of the opening, with the tension bands extending therebetween.

Once means have been put in place to apply a closing force across the cut, the insert is preferably moved a little further into the cut to ensure compressive and tensile forces are developed across the cut.

### BRIEF DESCRIPTION OF THE DRAWINGS

Embodiments of the present invention will now be described by way of example only and with reference to the accompanying drawings, in which:

FIG. 1 is a perspective view of an insert according to the invention;

FIG. 2 is a longitudinal cross sectional view of the insert of FIG. 1;

FIGS. 3 and 4 are front views showing a tibia upon which a method according to the invention is being carried out.

FIG. 5 is a medial side view of the tibia of FIG. 3;

FIG. 6 is a similar view to FIG. 5 but at a further point during the method;

FIG. 7 is a plan view of the tibia of FIG. 6;

FIG. 8 is a similar view to FIG. 7 but with a slightly different method being carried out on a different tibia;

FIGS. 9 and 10 are similar views to FIG. 4 showing a method carried out in a first version in FIG. 9 and a second version in FIG. 10 of the invention; and

FIG. 11 is a diagrammatic side view of a tibia showing a method being carried out according to a third version of the invention.

### DETAILED DESCRIPTION

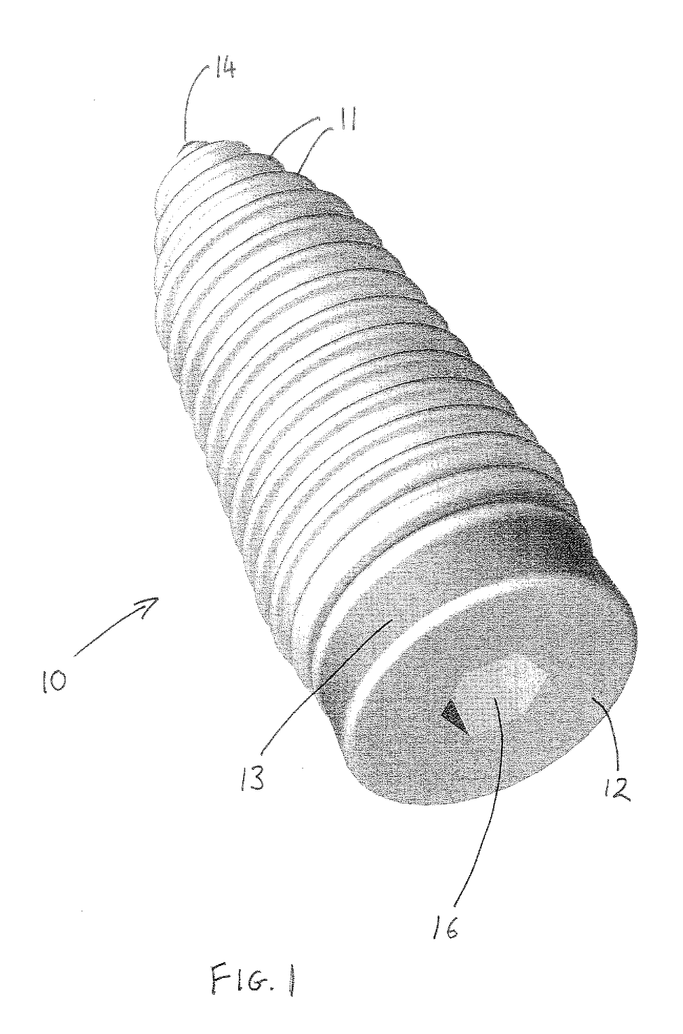

FIG. 1 shows an insert 10 according to an invention usable in an opening wedge osteotomy. The insert 10 is conical and may be made of an osteoconductive and bioabsorbable material. An external surface formation in the form of a helical rib 11 is provided on the insert 10. The height of the rib 11 reduces to zero short of the proximal end 12 of the insert 10 to provide an unribbed frusto conical section 13.

The distal end 14 of the insert 10 tapers to a greater degree than the remainder of the insert 10, to an approximate bell shape. An engagement formation in the form of a tapering hexagonal cross section opening 16 is provided extending for most of the length of the insert 10 from the proximal end 12.

The carrying out of an opening wedge osteotomy technique using the insert 10 of FIG. 1 will now be described. A medial opening wedge high tibial osteotomy (HTO) procedure was undertaken to correct angular deformity associated with unicompartmental arthritis of the knee. The surgical procedure was performed according to the following steps:

Obtain full-length, standing A/P and lateral radiographs.

Geometrically determine the degree of correction required to shift the mechanical axis to the point of intersection on the lateral side.

Perform skin incision with care to avoid the saphenous vein and its branches.

Reflect back the anterior portion of the medial collateral ligament.

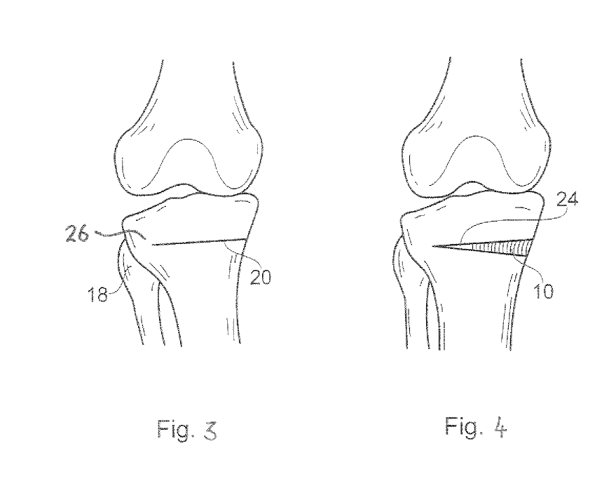

Drill two guide pins from the medial side to within 1 cm of the lateral cortex (angled towards the fibular head 18 (see FIG. 3)).

Confirm position by radiology.

Dissect anteriorly behind the patellar tendon and posteriorly with decortication if possible to preserve a periosteal sleeve.

Pass curved elevators to protect the patellar tendon anteriorly and the major vessels posteriorly.

Position an oscillating saw inferior to the guide pins to cut medial to lateral 20 taking great care not to penetrate the lateral cortex 26.

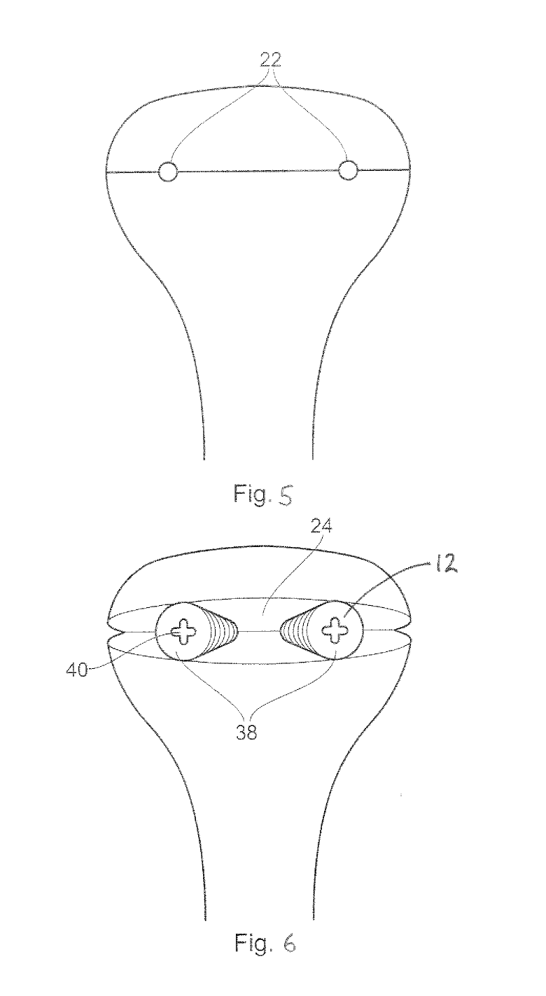

Drill two guide-holes 22 (see FIG. 5) in the plane of the cut extending 40 mm (approximately two thirds) from the medial cortex angled towards the fibular head 18.

Two inserts 10 are located in the guide holes 22 and are rotated by an appropriate tool (not shown) and with gentle valgus stress, into the guide-holes 22 to open the osteotomy. This is illustrated by FIGS. 3 and 4, and also FIGS. 5 and 6. In the latter slightly different inserts 38 are used with cruciform openings 40. Appropriate size inserts 10 are chosen relative to the degree of correction required. The proximal end 12 of the inserts should reside generally flush with the medial cortical surface when in their final position, taking care to maintain the lateral tibial cortex hinge. The degree of correction is then verified.

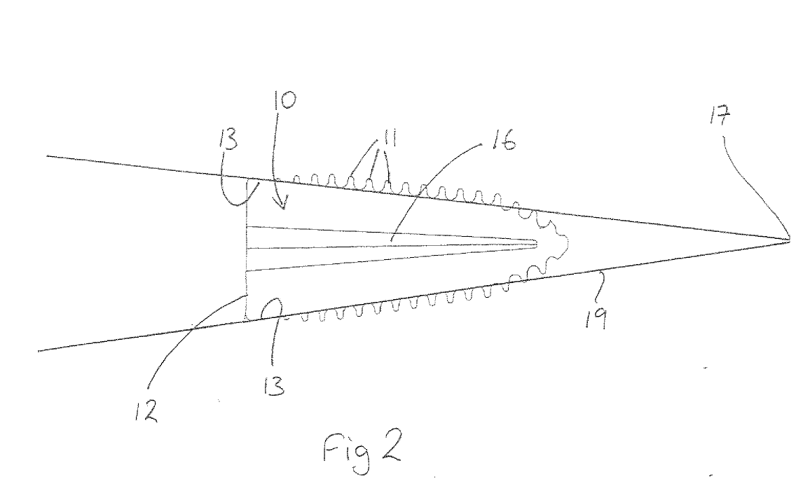

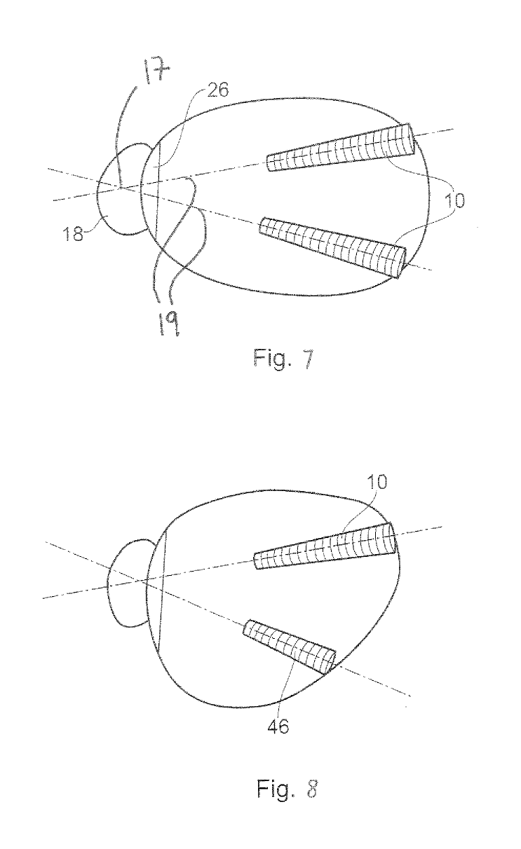

The unribbed section 13 engages against cortical bone while the ribs 11 engage cancellous bone when the insert 10 is fully inserted, helping to prevent extrusion of the inserts 10. In this procedure the apex 17 as illustrated by the lines 19 in FIGS. 2 and 7, of the main part of the insert 10 substantially coincides with the position of the lateral cortical hinge 26 (see FIG. 7).

An appropriate system is then used to provide a closing force across the osteotomy to compress the site and provide stability to the reconstruction, and details concerning this are provided below. The open wedge 24 produced is then filled with suitable bone graft. Preferably this graft adheres to the absorbable inserts 10 and this provides added stability. Preferred closure of wounds and post-operative management can then be followed.

The application of a compressive force e.g. by the use of a compression plate, across the osteotomy at the surface of the bone midway between the inserts 10 will lever the lateral cortical hinge into tension. The preferred situation, in order to provide maximum stability to the reconstruction, is where compression is applied at all three points represented by the cortical hinge 26 and the two areas of contact between the medial cortex and for each insert the unribbed sections 13 of the inserts. This is achieved when the tensile supporting elements of the system cross the central plane of the osteotomy within the triangle formed by these three points. This combination of tension and compression elements is a tensegrity structure.

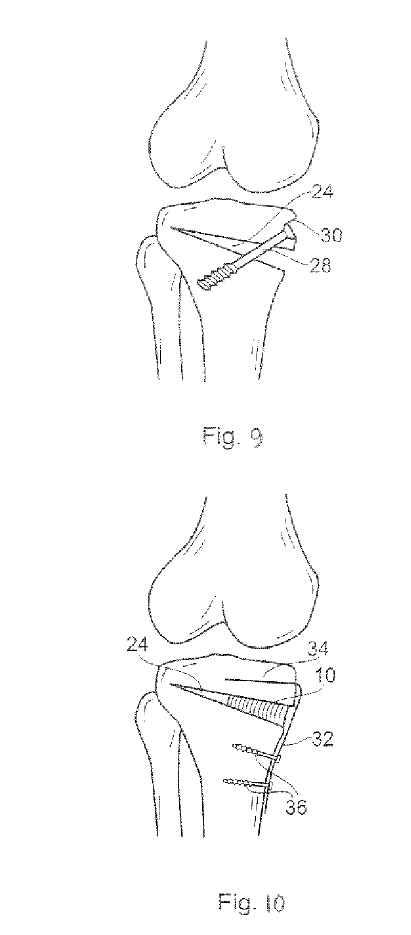

FIG. 9 shows one system of providing a compression force, with an inclined compression screw 28 extending across the wedge 24 to pass through said triangle. A notch/countersink 30 may be formed in the bone to receive the head of the screw 28.

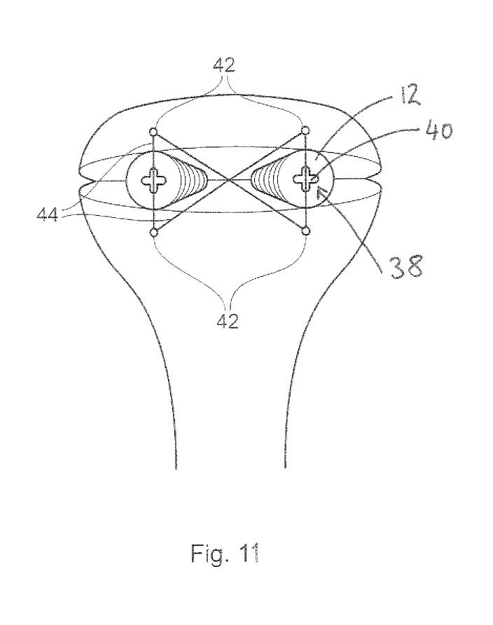

FIG. 10 shows an alternative system for providing a compression force with a plate 32 one end of which is located in a slot 34 in the bone above the wedge 24. The plate 32 extends across the open end of the wedge 24 and is held in position therebelow by two or more screws 36.

FIG. 11 shows a third system for providing a compression force. In this instance the inserts 38 are used having a cruciform opening 40 to permit rotation thereof. Here four pins 42 are provided in the bone, two above and two below the open end of the wedge 24. Tension bands 44 extend between the pins 42 in a bow tie shaped configuration.

In practice when a closing compression force has been applied, for instance, in any of the above ways, the inserts 10, 38 may be rotated a little further into the wedge 24 to increase both the compressive forces on the inserts, and the tensile forces on the tensile supporting elements, thus placing the system further in to tensegrity. The use of two inserts provides three points of support, the two inserts at their contact with the medial cortex and the lateral cortical hinge, giving an inherently more stable system than prior art devices and methods. The diameter of the guide holes may vary to accommodate different size inserts and to provide different degrees of correction.

It is to be realized that the method and apparatus used can be modified as particular situations dictate. For instance the guide holes can be of different respective dimensions. The inserts may have different diameters, lengths and also degrees of convergence. FIG. 8 shows for instance an arrangement which differs from that in FIG. 7, with one smaller insert 46. Such variations can provide correction for anterior/posterior as well as lateral/medial angulation, or where the medial-lateral distance from the apex of the osteotomy i.e. the cortical hinge 26, varies due to the shape of the bone, as shown in FIG. 8. The bone graft material may be used to fill the wedge gap between the inserts and external to the inserts between the cut bone ends.

There is thus described apparatus for, and also methods of, providing tensegrity in an opening wedge osteotomy system thereby conferring mechanical stability by the combined use of counteracting forces of compression and tension. The apparatus used is such that it can be inexpensively produced by conventional methods.

Various other modifications may be made without departing from the scope of the invention. For instance the inserts may take a different form or shape. They may be solid or porous and may contain through-holes or other surface features to aid osseointergration. The base of the cone may not be orthogonal to its vertical axis in order to allow for the guide hole(s) not being orthogonal to the bone surface. Different means for providing compression across the wedge may be used. The osteotomy may be performed on other bones in the body. The guide holes may be formed prior to the cut.

Whilst endeavoring in the foregoing specification to draw attention to those features of the invention believed to be of particular importance it should be understood that the Applicant claims protection in respect of any patentable feature or combination of features hereinbefore referred to and/or shown in the drawings whether or not particular emphasis has been placed thereon.

1. Apparatus for use in opening wedge osteotomy, the apparatus including an insert locatable in an opening in bone, the insert being elongate and having distal and proximal ends, the insert converging to its distal end, the insert having a helical external rib, and a guide formation at its proximal end to facilitate rotation thereof.

2. Apparatus according to claim 1, in which the insert is conical.

3. Apparatus according to claim 2, in which the insert tapers from its proximal end to its distal end, with the degree of tapering increasing towards the distal end.

4. Apparatus according to claim 1, in which the insert is frusto conical.

5. Apparatus according to claim 1, in which the insert is made of an osteoconductive material.

6. Apparatus according to claim 1, in which the insert is made of a bioabsorbable material.

7. Apparatus according to claim 1, in which the guide formation comprises a profiled opening in the proximal end of the insert.

8. Apparatus according to claim 1, in which the external rib finishes spaced from the proximal end of the insert.

9. Apparatus according to claim 1, in which the apparatus includes a pair of inserts.

10. Apparatus according to claim 1, in which the apparatus also includes gap closing means for applying a compressive force across the wedge gap.

11. Apparatus according to claim 10, in which the gap closing means includes a plate locatable across the opening of the wedge gap.

12. Apparatus according to claim 10, in which the gap closing means includes a screw extendable across the wedge gap.

13. Apparatus according to claim 10, in which the gap closing means includes tension bands extendable across the opening of the wedge gap.

14. Apparatus according to claim 13, in which fastening means are provided mountable on either side of the wedge gap, between which the tension bands extend.

15. A method of carrying out an opening wedge osteotomy, the method including forming a cut in the bone to produce a cut plane, forming two guide holes in the cut plane, locating in each guide hole an insert, each insert being elongate, having distal and proximal ends, converging to its distal end, having a helical external rib, and a guide formation at its proximal end to facilitate rotation thereof.

16. A method according to claim 15, in which the two guide holes are convergent.

17. A method according to claim 15, in which the closing force is provided by locating a plate across the opening of the cut.

18. A method according to claim 15, in which the closing force is provided by locating a screw extendible across the cut.

19. A method according to claim 18, in which the screw is inclined relative to the cut plane.

20. A method according to claim 15, in which the closing force is provided by extending tension bands across the opening.

21. A method according to claim 20, in which fastening means is mounted in the bone on either side of the opening, with the tension bands extending therebetween.

22. A method according to claim 15, in which once means have been put in place to apply a closing force across the cut, the insert is moved a little further into the cut to ensure compressive and tensile forces are developed across the cut.
